(19) United States
(12) Patent Application Publication
(10) Pub. No.: US 2007/0044249 A1
(43) Pub. Date: Mar. 1, 2007

(54) Methods and Kits for Touching up Hair Color

(76) Inventors: Donna Jean Lisowski, Watchung, NJ (US); Lou Ann Christine Vena, Scotch Plains, NJ (US)

Correspondence Address: JULIE BLACKBURN;REVLON CONSUMER PRODUCTS CORPORATION, 237 PARK AVENUE, NEW YORK, NY, 10017, US

(21) Appl. No.: 11/426,367

(22) Filed: Jun. 26, 2006

(51) Int. Cl. A61K 8/00

(52) U.S. Cl. 8/405

(57) ABSTRACT

An oxidative hair color kit for touching up hair between oxidative hair color procedures comprising at least one first receptacle suitable for mixing and applying an oxidative hair dye mixture prepared by combining and oxidative dye composition and an aqueous oxidizing agent compositions, containing at least one of the oxidative dye composition or the aqueous oxidizing agent composition but not both, at least one second receptacle containing the composition not found in the first receptacle, and at least one applicator that can be affixed to the first receptacle for use in applying the contents of the receptacle to the hair; and a method for touching up the hair between hair color procedures.

### TECHNICAL FIELD

The invention is in the field of kits, methods, and components for use in oxidatively coloring hair, specifically, for touching up faded hair strands or new hair growth between hair coloring procedures.

### BACKGROUND OF THE INVENTION

About fifty nine percent of the female population colors their hair, with about half of those using salons and the other half coloring their hair at home using kits purchased at retail. In most cases the hair is colored to cover gray. In a smaller percentage of cases the user simply desires to change the color of her hair.

Oxidative, or permanent hair color, which permanently changes the color of the hair, is most often used by consumers. However, because hair grows about ¼ to ½ inch per month, new hair growth becomes evident at the hair roots in about two weeks. In addition, hair that is oxidatively colored may fade first around the temples revealing visible gray or originally colored strands. For this reason, consumers who color their hair with permanent color generally repeat the procedure every four to six weeks so that new hair growth can be colored and any faded strands can be re-colored. Even with repeat procedures every four to six weeks, fading and new hair growth at the roots are still evident starting at about two weeks thereafter, particularly in consumers who are predominantly gray. Hair color users find it enough of a burden to recolor their hair every four to six weeks, much less having to worry about coloring more often than that to touch up new hair growth or faded hair strands.

Currently hair color users have a variety of options for touching up hair between oxidative coloring procedures. Some well heeled consumers simply visit the salon more often. When the hair is oxidatively dyed on a regular basis the colorant is applied to the roots and new hair growth for the standard period of time, followed by a quick five minute application of color all over at the end of the procedure. This provides a nice result but repeated salon visits are expensive.

Another option for hair color users who wish to touch up are the various color conditioners or shampoos that are sold in salons and drugstores. Such products generally contain either vegetable or semi-permanent direct dyes. The term “direct dye” means a dye that is operable to color hair when directly applied to the hair and without the need for activating with an oxidizing agent. When in the shampoo form, the composition is used to shampoo the hair. When in the conditioner form, the conditioners are applied to the hair after shampooing, just like a normal hair conditioner. The dyes present in the shampoo and/or conditioner are said to boost color. However, such products are developed for use on the whole head, not just new hair growth or faded hair strands. If hair color users wish to treat only select strands of hair with the color conditioner they must fashion their own applicator and process.

A root touch up kit sold by Clairol under the name “Nice n' Easy Permanent Root Touch Up” is commercially available in many mass market outlets. The kit contains one receptacle containing an oxidative dye composition, a second receptacle containing an aqueous oxidizing agent composition, a mixing tray, and an applicator. The user combines the oxidative dye composition and oxidizing agent composition in the mixing tray, then applies it to the hair with a rake-like applicator. While this is an effective and popular product, it is messy to use and fraught with error potential. Drips and spills are common when the mixture in the mixing tray is applied to the hair with the applicator. There is also much waste because the components are mixed in the tray, then transferred to the applicator. The requirement for mixing the composition in a separate tray, then applying it to the hair complicates the processes. There is the potential that some users will not properly combine the ingredients, or spill one or more of the components that are combined to prepare the oxidative dye mixture.

Accordingly, there is a need for a simple, consumer friendly, inexpensive method and kit for consumers to touch up their hair color between oxidative coloring procedures which eliminates the drawbacks mentioned above. The method, compositions, and kit components should be designed to treat select hair strands to touch up color in a short period of time and the product should be storage stable. In addition, the mixture should be applied to the hair in an applicator that also serves as a mixing receptacle for the oxidative dye and aqueous oxidizing agent, to thereby minimize complications.

It is an object of the invention to provide a method for touching up oxidatively colored hair between oxidative dyeing procedures, specifically to touch up new hair growth or faded areas, so that the consumer does not have to oxidatively color her hair as often.

It is a further object of the invention to provide a kit for purchase by consumers who wish to touch up their hair using permanent hair color.

It is a further object of the invention to provide a method for oxidatively touching up hair between oxidative coloring procedures by treating the new hair growth or faded hair strands with an oxidative touch up composition.

It is a further object of the invention to provide a kit for oxidatively touching up hair between oxidative coloring procedures by treating new hair growth or faded hair strands, wherein the oxidative dye composition and aqueous oxidizing agent composition are combined and mixed in the container that serves as the applicator to apply the mixture to the hair.

It is a further object of the invention to provide a kit and container for oxidatively touching up faded hair strands and new hair growth between oxidative hair color procedures with a container and applicator that is inexpensive, easy to use, and provides precise application.

It is a further object of the invention to provide a container with a sponge tip for oxidatively touching up faded hair strands and new hair growth between oxidative dye procedures that provides precise application and which does not drip product and reduces opportunities for mess.

### SUMMARY OF THE INVENTION

The invention comprises an oxidative hair color kit for touching up hair between oxidative hair color procedures comprising at least one first receptacle suitable for mixing and applying an oxidative hair dye mixture prepared by combining and oxidative dye composition and an aqueous oxidizing agent composition, containing at least one of the oxidative dye composition or the aqueous oxidizing agent composition but not both, at least one second receptacle containing the composition not found in the first receptacle, and at least one applicator affixable to the first receptacle for use in applying the contents of the receptacle to the hair.

The invention further comprises a method for touching up hair between oxidative hair color procedures comprising forming an oxidative dye mixture by combining an oxidative dye composition and an aqueous oxidizing agent composition in a receptacle, attaching a sponge tip to the receptacle, and dispensing the mixture to the hair to be touched up through the sponge tip.

### BRIEF DESCRIPTION OF THE DRAWINGS

FIG. 1: depicts the general configuration of one type of a suitable sponge tip that may be used as an applicator for the hair dye mixture. This type of sponge tip is most suitable when the hair dye mixture is more viscous in consistency.

FIG. 2: depicts the sponge tip applicator of FIG. 1 that is seated within cap.

FIG. 3: depicts another type of suitable sponge tip that may be used as the applicator for the hair dye mixture.

FIG. 4: depicts the sponge tip of FIG. 3 seated within cap.

FIG. 5: illustrates the various components that may be found in the kit of the invention.

FIG. 6: is an exploded view of the container with sponge tip for affixing to container.

FIG. 7: shows how the container with sponge tip applicator is used to touch up the hair with the hair dye mixture.

### DETAILED DESCRIPTION

I. The Container with Sponge Tip

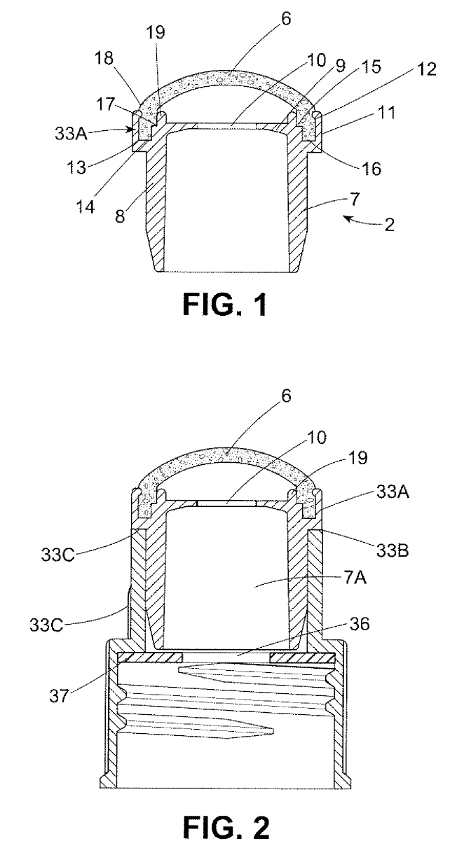

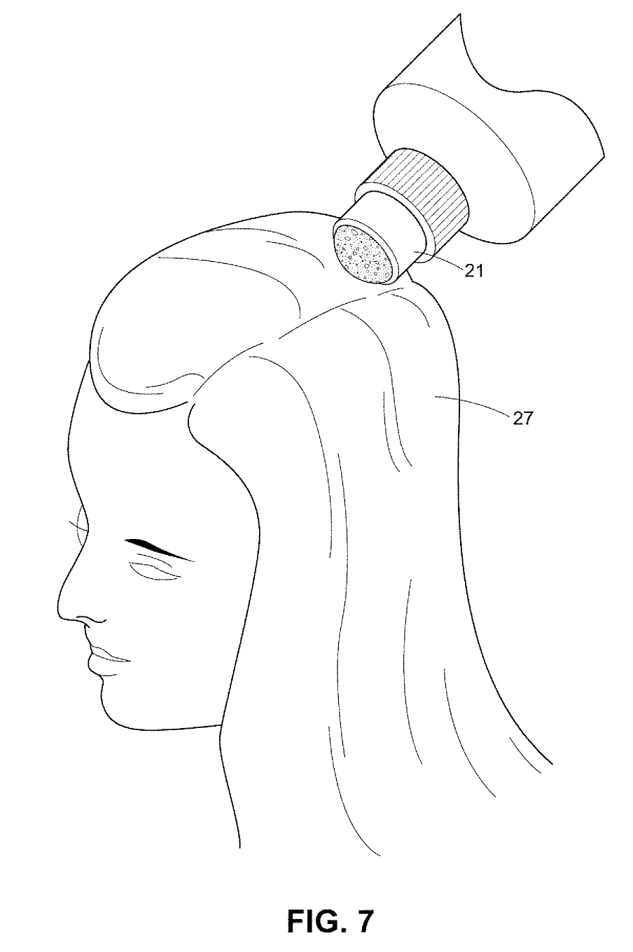

The container 1 with sponge tip 2 (see FIG. 4), in one embodiment, is depicted in FIGS. 1, 2, and 7.

A. Container

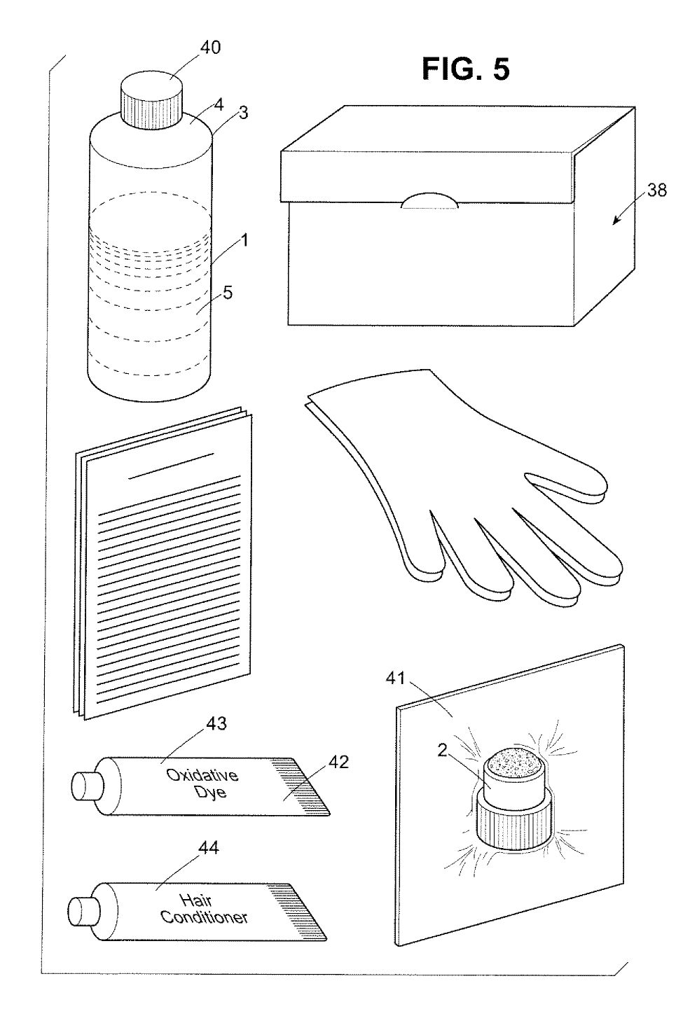

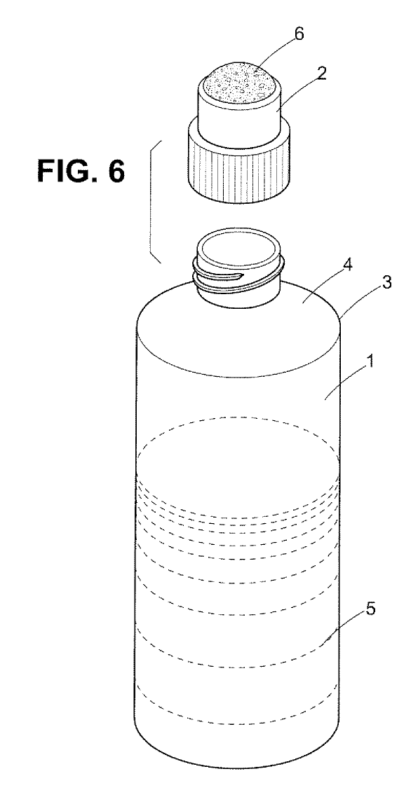

The container 1 is illustrated in FIGS. 5, 6, and 7. The container 1 is generally in the form of a bottle 3 preferably having shoulders 4 that may be rounded or square. Container 1 may be used to contain either the oxidative dye composition or the aqueous oxidizing agent composition (both described in further detail below). The sponge tip 2 is affixed to the container 1 and the mixture that is formed within container 1 by adding one of the compositions required to form the oxidative dye mixture to the container 1. The oxidative dye mixture is then used to color the hair. It is noted that when oxidatively coloring hair, the hair dye composition that contains the dyes is not operable by itself to color hair. Rather, it must be activated by combining the dye with an oxidizing agent such as hydrogen peroxide. The combination of the hair dye composition and the aqueous hydrogen peroxide composition will form a hair dye mixture that is operable to color hair.

Preferred is where the container 1 in the kit is used to contain the aqueous oxidizing agent composition 5. Then, the oxidative hair dye composition 43 that is found in a separate receptacle is added to aqueous oxidizing agent composition 5 in the container 1 and mixed to form a hair dye mixture that is used to touch up the hair.

The container 1 where the aqueous oxidizing agent composition 5 is stored may be made of a variety of materials. Preferred is where container 1 is made of thermoplastic polymeric material such as polyethylene or polypropylene. Most preferred is where the container is made of polyethylene, in particular high density polyethylene. Most preferred is high density polyethylene having one or more of the following specifications:

| | | | | | Measured by ASTM |
|---|---|---|---|---|---|
| Specification | Broad Range | Middle Range | Preferred Range | Units | Test Method |
| Melt Index | 0.1 to 1.0 | 0.2 to 0.9 | 0.3 to 0.8 | grams/10 | D1238 |
| | | | | minutes | |
| Density | 0.5 to 2.0 | 0.6 to 1.8 | 0.7 to 1.2 | grams/cubic | D1505 |
| | | | | centimeter | |
| Tensile strength | 2,500 to 6,000 | 3,000 to 5,000 | 3,500 to 4,500 | pounds per | D638 |
| at yield | | | | square inch | |
| Elongation at break | less than 500 | | | % | D638 |
| Flexural modulus | 100,000 to 300,000 | 130,000 to 250,000 | 150,000 to 200,000 | pounds per | D790 |
| | | | | square inch | |
| Tensile impact | 100 to 200 | 115 to 175 | 120 to 150 | ft-lb/in | D1822 |
| Low temperature | less than −76 | | | ° C. | D746 |
| brittleness, F. 50 | | | | | |
| Heat deflection | 25 to 125 | 40 to 100 | 50 to 90 | ° C. | D648 |
| temperature at 66 psi | | | | | |
| Vicat softening point | 75 to 175 | 100 to 150 | 110 to 140 | ° C. | D1525 |
| Shore D Hardness | 25 to 150 | 35 to 100 | 40 to 80 | | D2240 |

The preferred polyethylene for use in making the container is made by Equistar and has the trade name Petrothene® LR 7340, high density polyethylene, blow molding grade, having a melt index of 0.38, density 0.953, tensile strength @ yield of 4,000, elongation @ break of less than 500, flexural modulus 176,000; tensile impact 131; low temperature brittleness F50 of less than −76; heat deflection temperature at 66 psi of 75; Vicat softening point of 127; and Shore D hardness of 67, in the units and using the ASTM test methods mentioned above.

The container 1 may be made by blow molding or other techniques typically used in the container manufacturing art.

B. The Sponge Tip

FIGS. 1 and 2 depict one embodiment of the sponge tip 2, which is one preferred applicator for the mixture. Sponge tip 2 comprises a sponge 6. The sponge may be made of typical thermoplastic polymeric materials such as polyethylene, polystyrene, polyurethane, silicone, and the like. Preferred is where the sponge is made of open celled foam, that is foam where the cells are open to permit interconnecting between them such that the mixture can filter through the channels onto the hair to be treated. Sponge 6 is preferably in the form of a flat sheet of open celled foam having a thickness ranging from about 0.05 to 0.8 inches.

Sponge 6 is affixed to housing 7. Housing 7 comprises legs 8 extending from top table surface 9 having an aperture 10. Sponge 6 is affixed to housing 7 by capturing sponge edges 11 in depressions 12 that are found on either side of surface 9 and generally at the top of legs 8. The depressions 12 preferably have side edges 13, bottom edges 14, and inner side edges 15. Most preferably inner side edges 15 are in stepwise configuration providing a narrower base portion 16 and a wider top portion 17. Preferably, the top side edge 13 of depression 12 has a slight hook 18 that further assists in securing sponge 6 to housing 7. On the top inner surface of legs 8 a slight protrusion 19 extends above surface 9 and further assists in securing sponge 6 to housing 7. Sponge 6 is preferably in the form of a flat sheet with a thickness ranging from about 0.1 to about 0.5 inches.

FIG. 2 depicts housing 7 seated into cap 33. Specifically, external upper wall 33A of housing 7 is in the form of a step 33B. Step 33B abuts top outer edge of cap 33 top wall 33C and effectively seats housing 7 in cap 33. When sponge tip 2 is secured to container 1, the cosmetic within container 1 is capable of flowing through second aperture 36, through inner housing chamber 7A, through aperture 10, onto sponge 6, then on to the hair. Supports 37 may serve to narrow second aperture 36 and further regulate flow to ensure that it is appropriate for the composition that is being applied to the hair.

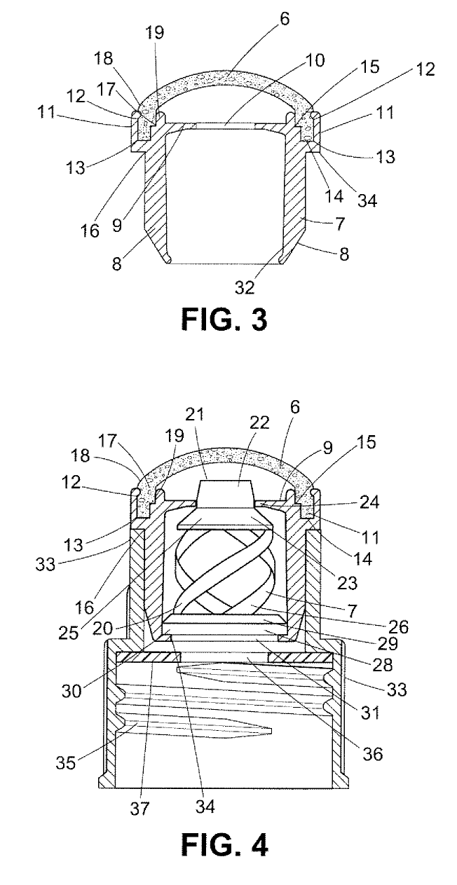

FIGS. 3 and 4 depict a second embodiment of sponge tip 2, which contains a compressible valve 20 secured into housing 7 to further regulate flow. Compressible valve 20 contains a head 21 with a neck 22 and shoulders 23. Neck 22 is of a size and shape to fit into aperture 10 such that the base 24 of neck 22 fits within aperture 10 to close it so that fluid from the container cannot flow through aperture 10 when neck 22 is in place. Shoulders 23 provide a extending skirt 25 from neck base 24. Attached to shoulders are spring coils 26 that are compressible such that when the sponge 6 is placed into contact with the hair to be treated as depicted in FIG. 6, and the sponge is compressed against the hair to be treated 27. Compression against the hair to be treated 27, causes the neck 22 of compressible valve 20 to compress spring coils 26 as neck 22 moves downwardly and causes aperture 10 to open. Spring coils 26 are affixed to a base 28, which has a width sufficient to abut legs 8 of housing 7. More particularly, base 28 comprises a first base section 29 to which spring coils 26 are affixed. Second base section 30 is slightly larger in circumference than first base section 29 and serves to anchor compressible valve 20 in housing 7. Third base section 31 is of a slightly smaller circumference than second base section 30 and fits into space 32 between legs 8 of housing 7 such that third base section 31 is secured therein.

Housing 7 containing compressible valve 20 is further secured in cap 33 as described in the first embodiment. Cap preferably has a threaded inner surface 35 with a second aperture 36 that permits flow of fluid from container 1 into sponge tip 2. Supports 37 abut cap inner surface 35 and second aperture 36. Threaded inner surface 35 of cap 33 is designed to be affixed to neck of container 1 having similar mating engagements.

Either embodiment of sponge tip 2 is suitable. In some cases, the first embodiment may be more suitable if the hair dye mixture to be applied to hair is more viscous; typically the more recent drip-free formulas. On the other hand, if the hair dye mixture to be applied to hair is less viscous, the second embodiment of sponge tip 2 may be more suitable because compressible valve 20 opens and closes aperture 10 and regulates flow of the hair dye mixture.

II. The Kit

The kit used to oxidatively touch up hair as a variety of components, as depicted in FIG. 3.

The kit 38 generally comprises a container that is suitable for storing the various components found within the kit 38. The kit may be made of plastic, cardboard, paper, or a variety of other materials. Preferably the kit is made of cardboard or paper which easily lends itself to printing of graphics and indicia thereon. Examples of suitable graphics and indicia include model photographs, color chips showing the color to be obtained when using the product found within the kit, ingredient listings for the products found in the kit, the UPC Code, manufacturer information, and the like.

Also found in kit 38 is container 1 as described above in Section I.A, and sponge tip applicator 2 as described in Section I.B. In the preferred kit 38 container is closed by service cap 40 which is a plastic cap that closes container 1 that, preferably, contains the aqueous oxidizing agent composition. Sponge tip applicator 2 is found in a plastic bag 41 which the consumer opens when ready to use.

Kit contains the oxidative dye composition 42 that is combined with the aqueous oxidizing agent composition to form the oxidative dye mixture used for touching up hair. Oxidative dye composition 42 is preferably found in a tube 43 that is of a material that is suitable to stably preserve and contain oxidative dye composition. If desired, kit 38 may contain ancillary components such as hair conditioner 44, or gloves and instructions (not shown).

III. The Method

When the consumer desires to touch up the hair, the kit 38 is opened. Service cap 40 is removed from container 1. The oxidative dye composition 42 is poured into container and mixed to form the oxidative dye mixture. The consumer may reapply service cap during the mixing. Sponge tip applicator 2 is removed from plastic bag 41, and the consumer secures sponge tip applicator 2 to container 1, as best depicted in FIG. 4. Then container 1 with secured sponge tip applicator 2 is used to apply the oxidative hair color mixture to the desired strands of hair as depicted in FIG. 6.

When sponge tip 2 is compressed against the hair strands to be treated the sponge 6 is compressed against hair 27, permitting the hair dye mixture to flow through apertures 36 and 10 onto hair. Alternatively, if the second embodiment of sponge tip is used, the neck 22 is compressed against spring coils 26 causing aperture 10 to open, permitting the hair dye mixture to flow from container 2 through housing 7 and aperture 10 and be applied to hair. When the consumer no longer compresses the sponge tip against the hair, neck 22 moves in to plug aperture 10 and prevent the hair color mixture from exuding from it.

The oxidative dye mixture is applied to the desired strands of hair using container 1 with sponge applicator 2. When the consumer has finished, the unit is discarded because the oxidative dye mixture loses effectiveness after a fairly short period of time.

The oxidative dye mixture is left on the hair for a period of time ranging from 1 to 60, preferably from about 2 to 30, more preferably from about 3 to 12 minutes, most preferably about 10 minutes, then rinsed off with water. The treated hair strands will become colored in a color that blends with the existing color of the hair strands that were not treated.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. An oxidative hair color kit for touching up hair between oxidative hair color procedures comprising at least one first receptacle suitable for mixing and applying an oxidative hair dye mixture prepared by combining and oxidative dye composition and an aqueous oxidizing agent composition, containing at least one of the oxidative dye composition or the aqueous oxidizing agent composition but not both, at least one second receptacle containing the composition not found in the first receptacle, and at least one applicator affixable to the first receptacle for use in applying the contents of the receptacle to the hair.

2. The kit of claim 1 wherein the first receptacle contains the aqueous oxidizing agent composition.

3. The kit of claim 2 wherein the second receptacle contains the oxidative dye composition.

4. The kit of claim 3 additionally containing instructions.

5. The kit of claim 3 additionally containing rubber gloves.

6. The kit of claim 1 wherein the first receptacle is made of a thermoplastic polymeric composition.

7. The kit of claim 6 wherein the thermoplastic polymeric composition is polethylene or polypropylene.

8. The kit of claim 7 wherein the thermoplastic polymeric composition is high density polyethylene.

9. The kit of claim 8 wherein the high density polyethylene has one or more of the following specifications:

| | | | | | Measured by ASTM |
|---|---|---|---|---|---|
| Specification | Broad Range | Middle Range | Preferred Range | Units | Test Method |
| Melt Index | 0.1 to 1.0 | 0.2 to 0.9 | 0.3 to 0.8 | grams/10 | D1238 |
| | | | | minutes | |
| Density | 0.5 to 2.0 | 0.6 to 1.8 | 0.7 to 1.2 | grams/cubic | D1505 |
| | | | | centimeter | |
| Tensile strength | 2,500 to 6,000 | 3,000 to 5,000 | 3,500 to 4,500 | pounds per | D638 |
| at yield | | | | square inch | |
| Elongation at break | less than 500 | | | % | D638 |
| Flexural modulus | 100,000 to 300,000 | 130,000 to 250,000 | 150,000 to 200,000 | pounds per | D790 |
| | | | | square inch | |
| Tensile impact | 100 to 200 | 115 to 175 | 120 to 150 | ft-lb/in | D1822 |
| Low temperature | less than −76 | | | ° C. | D746 |
| brittleness, F. 50 | | | | | |
| Heat deflection | 25 to 125 | 40 to 100 | 50 to 90 | ° C. | D648 |
| temperature at 66 psi | | | | | |
| Vicat softening point | 75 to 175 | 100 to 150 | 110 to 140 | ° C. | D1525 |
| Shore D Hardness | 25 to 150 | 35 to 100 | 40 to 80 | | D2240 |

10. The kit of claim 1 wherein the first receptacle contains a sponge tip applicator.

11. The kit of claim 10 wherein the sponge tip applicator comprises a sponge, a housing having legs, and an aperture.

12. The kit of claim 11 wherein a compressible valve is secured in housing.

13. The kit of claim 12 wherein compressible valve contains a head with neck and shoulders, compressible spring coils, and a base.

14. The kit of claim 13 wherein the neck of compressible valve is of a size and shape sufficient to close aperture.

15. The kit of claim 11 wherein sponge tip applicator is secured into a cap.

16. The kit of claim 1 wherein the first receptacle is made of high density polyethylene having melt index ranging from 0.1 to 1.0 grams/10 minutes, a density of 0.5 to 2.0 grams/cubic centimeter, and a Shore D Hardness of 25 to 150.

17. The kit of claim 1 wherein the aqueous oxidizing agent composition comprises hydrogen peroxide.

18. The kit of claim 1 further comprising a receptacle containing a hair conditioner.

19. A method for touching up hair between oxidative hair color procedures comprising forming an oxidative dye mixture by combining an oxidative dye composition and an aqueous oxidizing agent composition in a receptacle, attaching a sponge tip to the receptacle, and dispensing the mixture to the hair to be touched up through the sponge tip.

20. The method of claim 19 wherein the hair to be touched up is new hair growth or faded or discolored strands of hair.
